(19) United States
(12) Patent Application Publication
(10) Pub. No.: US 2005/0112668 A1
(43) Pub. Date: May 26, 2005

(54) Methods of treating fat metabolism disorders

(75) Inventor: Ying-Hue Lee, San Trun City (TW)

Correspondence Address: FISH & RICHARDSON PC, 225 FRANKLIN ST, BOSTON, MA, 02110, US

(73) Assignee: Academia Sinica, a Taiwan institution

(21) Appl. No.: 10/981,237

(22) Filed: Nov. 4, 2004

Related U.S. Application Data
(62) Division of application No. 10/211,423, filed on Aug. 2, 2002, now abandoned.

(51) Int. Cl. C12Q 1/68; A61K 48/00

(52) U.S. Cl. 435/6; 514/44

(57) ABSTRACT

A method of determining whether a subject is suffering from or at risk for developing a disorder in fat metabolism. The method includes providing a sample from the subject and determining a Gₛα expression level in the sample. If the Gₛα expression level in the sample is different from that in a sample from a normal subject, it indicates that the subject is suffering from or at risk for developing a disorder in fat metabolism. Also disclosed are a method of identifying a compound for treating a disorder in fat metabolism, and a method and a composition for treating such a disorder.

### RELATED APPLICATIONS

This application is a divisional application and claims priority to U.S. application Ser. No. 10/211,423, filed on Aug. 2, 2002, the contents of which are incorporate herein in their entirety.

### BACKGROUND

The heterotrimeric G proteins (α, β and γ subunits) locate at the cytoplasmic side of the plasma membrane, where they interact with transmembrane heptahelical receptors for hormones (such as β-adrenergic hormone) and neurotransmitters (Neer (1995) Cell 80, 249-257). The monomeric Gₛα, once activated by binding to GTP, dissociates from the dimeric βγ subunit. The dissociated and GTP-bound Gₛα then stimulates effector proteins such as adenylyl cyclase to produce the secondary messenger cAMP to elicit cellular responses including cell growth (Hepler and Gilman (1992) Trends Biochem Sci 17, 383-387).

### SUMMARY

This invention relates to the use of a G protein stimulatory a subunit (Gₛα) gene in diagnosing and treating a disorder in fat metabolism, and in identifying therapeutic compounds for treating such a disorder.

A “disorder in fat metabolism” is a disorder associated with abnormal (i.e., below or above the normal level) fat metabolism. Obesity is one example of such disorders.

In one aspect, this invention features a method of determining whether a subject is suffering from or at risk for developing a disorder in fat metabolism. In one example, the method includes providing a sample from a subject and determining a Gₛα expression level in the sample. If the Gₛα expression level in the sample is different from that in a sample from a normal subject, it indicates that the subject is suffering from or at risk for developing a disorder in fat metabolism. The sample can be prepared from a fat tissue biopsy, e.g., a white adipose tissue sample. The Gₛα expression level can be determined by measuring the amount of the Gₛα mRNA, or the Gₛα protein itself. The Gₛα mRNA level can be determined, for example, by in situ hybridization, PCR, or Northern blot analysis. The Gₛα protein level can be determined, for example, by Western blot analysis. In another example, the method includes providing a sample from a subject and determining a Gₛα activity level in the sample. If the Gₛα activity level in the sample is different from that in a sample from a normal subject, it indicates that the subject is suffering from or at risk for developing a disorder in fat metabolism. The Gₛα activity can be determined, e.g., by measuring its binding to the βγ subunit of a G protein, activation of effectors, or targeting to a membrane.

In another aspect, this invention features a method of identifying a compound for treating a disorder in fat metabolism. In one example, the method includes contacting a compound with a cell (e.g., a fat cell such as a white adipose cell) and determining a Gₛα expression level in the cell. If the Gₛα expression level in the presence of the compound is different from that in the absence of the compound, the compound is a candidate for treating a disorder in fat metabolism. In another example, the method includes contacting a compound with a cell and determining a Gₛα activity level in the cell. If the Gₛα activity level in the presence of the compound is different from that in the absence of the compound, the compound is a candidate for treating a disorder in fat metabolism.

In still another aspect, this invention features a method of treating a disorder in fat metabolism. The method includes identifying a subject suffering from or being at risk for developing a disorder in fat metabolism and administering to the subject a composition to increase a Gₛα level in the subject. The composition can contain a nucleic acid encoding a Gₛα protein, or a Gₛα protein itself. The “Gₛα protein” refers to both the wild-type Gₛα protein and its variants with an equivalent biological function (e.g., a fragment of the wild-type Gₛα protein). The composition can be administered directly to the fat tissue of a subject (e.g., the white adipose tissue).

Also within the scope of this invention is a pharmaceutical composition for treating a disorder in fat metabolism. The composition can contain a nucleic acid encoding a Gₛα protein and a pharmaceutically acceptable carrier. Alternatively, it can contain a Gₛα protein itself and a pharmaceutically acceptable carrier.

The present invention provides methods for diagnosing and treating a disorder in fat metabolism associated with abnormal expression of the Gₛα gene. The details of one or more embodiments of the invention are set forth in the accompanying description below. Other features, objects, and advantages of the invention will be apparent from the detailed description, and from the claims.

### DETAILED DESCRIPTION

The present invention is based on an unexpected discovery that Gₛα plays an important role in fat metabolism. As demonstrated in the examples below, Gₛα is up-regulated specifically in the white adipose tissue (WAT) of C/EBPαβ mice, where the mitochondria content and energy oxidation are specifically and significantly increased. In addition, over-expression of human or mouse Gₛα gene in human or mouse fat cells not only effectively inhibits fat accumulation, but also efficiently consumes fat that has been stored in the cells.

This invention provides methods for diagnosing and treating fat metabolism disorders associated with abnormal expression of the Gₛα gene, and identifying therapeutic compounds for treating such disorders.

A diagnostic method of this invention involves comparing a Gₛα gene expression level or Gₛα protein activity level in a sample (e.g., a fat tissue biopsy such as a white adipose tissue sample) prepared from a subject with that in a sample prepared from a normal person, i.e., a person who does not suffer from a fat metabolism disorder. A lower or higher Gₛα expression or activity level indicates that the subject is abnormal in fat metabolism. The methods of this invention can be used on their own or in conjunction with other procedures to diagnose fat metabolism disorders in appropriate subjects.

The Gₛα expression level can be determined at either the MRNA level or at the protein level. Methods of measuring mRNA levels in a tissue sample are known in the art. In order to measure mRNA levels, cells can be lysed and the levels of Gₛα mRNA in the lysates or in RNA purified or semi-purified from the lysates can be determined by any of a variety of methods including, without limitation, hybridization assays using detectably labeled Gₛα-specific DNA or RNA probes and quantitative or semi-quantitative RT-PCR methodologies using appropriate Gₛα-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

Methods of measuring protein levels in a tissue sample or a body fluid are also known in the art. Many such methods employ antibodies (e.g., monoclonal or polyclonal antibodies) that bind specifically to a Gₛa protein. In such assays, the antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to bodily fluids or to lysates of cells, and others (e.g., immunohistological methods or fluorescence flow cytometry) applied to histological sections or unlysed cell suspensions. Methods of measuring the amount of label will be depend on the nature of the label and are well known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

The Gₛα activity can be determined by methods well known in the art, e.g., by measuring its binding to the βγ subunit, activation of effectors, and targeting to a membrane (Evanko, et al. (2000) J. Biol. Chem 275, 1327-1336).

This invention also provides a method for identifying candidate compounds (e.g., proteins, peptides, peptidomimetics, peptoids, antibodies, small molecules or other drugs) that increase the Gₛα gene expression level or Gₛα protein activity level in a cell (e.g., a fat cell such as a white adipose cell). Compounds thus identified can be used to treat conditions characterized by abnormal Gₛα activity, e.g., fat metabolism disorders.

The candidate compounds of the present invention can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann, et al. (1994) J. Med. Chem. 37, 2678-85; and Lam (1997) Anticancer Drug Des. 12, 145.

Examples of methods for the synthesis of molecular libraries can be found in the art, for example, in: DeWitt, et al. (1993) PNAS USA 90, 6909; Erb, et al. (1994) PNAS USA 91, 11422; Zuckermann, et al. (1994) J. Med. Chem. 37, 2678; Cho, et al. (1993) Science 261, 1303; Carrell, et al. (1994) Angew. Chem. Int. Ed. Engl. 33, 2059; Carell, et al. (1994) Angew. Chem. Int. Ed. Engl. 33, 2061; and Gallop, et al. (1994) J. Med. Chem. 37,1233.

Libraries of compounds may be presented in solution (e.g., Houghten (1992) Biotechniques 13, 412-421), or on beads (Lam (1991) Nature 354, 82-84), chips (Fodor (1993) Nature 364, 555-556), bacteria (Ladner, U.S. Pat. No. 5,223,409), spores (Ladner U.S. Pat. No. 5,223,409), plasmids (Cull, et al. (1992) PNAS USA 89, 1865-1869), or phages (Scott and Smith (1990) Science 249, 386-390; Devlin (1990) Science 249, 404-406; Cwirla, et al. (1990) PNAS USA 87, 6378-6382; Felici (1991) J. Mol. Biol. 222, 301-310; and Ladnersupra.).

To identify compounds that modulate the Gₛα gene expression level or Gₛα protein activity level in a cell, a cell (e.g., a fat cell such as a white adipose cell) is contacted with a candidate compound and the expression level of the Gₛα gene or the activity level of the Gₛα protein is evaluated relative to that in the absence of the candidate compound. The cell can be a cell that contains the Gₛα gene yet does not naturally expresses it, a cell that naturally expresses Gₛα, or a cell that is modified to express a recombinant nucleic acid, for example, having the Gₛα promoter fused to a marker gene. The level of the Gₛα gene expression or the marker gene expression and the level of the Gₛα protein activity or the marker protein activity can be determined by methods described above and any other methods well known in the art. When the expression level of the Gₛα gene or the marker gene or the activity level of the Gₛα protein or the marker protein is lower or higher in the presence of the candidate compound than that in the absence of the candidate compound, the candidate compound is identified as a potential drug for treating a fat metabolism disorder.

This invention also provides a method for treating a fat metabolism disorder. Subjects to be treated can be identified, for example, by determining the Gₛα gene expression level or the Gₛα protein level in a sample prepared from a subject by methods described above. If the Gₛα gene expression level or the Gₛα protein level is lower or higher in the sample from the subject than that in a sample from a normal person, the subject is a candidate for treatment with an effective amount of compound that modulates the Gₛα level in the subject.

The treatment method can be performed in vivo or ex vivo, alone or in conjunction with other drugs or therapy.

In one in vivo approach, a therapeutic composition (e.g., a composition containing a compound that modulates the Gₛα gene expression level or the Gₛα protein activity level in a cell or a Gₛα protein itself) is administered to the subject. Generally, the compound will be suspended in a pharmaceutically-acceptable carrier (e.g., physiological saline) and administered orally or by intravenous infuision, or injected or implanted subcutaneously, intramuscularly, intrathecally, intraperitoneally, intrarectally, intravaginally, intranasally, intragastrically, intratracheally, or intrapulmonarily. For treatment of a fat metabolism disorder, the compound can be delivered directly to the fat tissue (e.g., the white adipose tissue).

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Alternatively, a polynucleotide containing a nucleic acid sequence encoding a Gₛα protein or a sequence complementary thereof can be delivered to the subject, for example, by the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al. (1995) J. Mol. Med. 73, 479). Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the Gₛα protein or a sequence complementary thereof is operatively linked to a promoter or enhancer-promoter combination. Enhancers provide expression specificity in terms of time, location, and level. Unlike a promoter, an enhancer can function when located at variable distances from the transcription initiation site, provided a promoter is present. An enhancer can also be located downstream of the transcription initiation site.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., an increased or decreased Gₛα level) in a treated subject. As is well known in the medical arts, the dosage for any one subject depends upon many factors, including the subject's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

An ex vivo strategy for treating subjects with a fat metabolism disorder associated with inadequate Gₛα activity can involve transfecting or transducing cells obtained from the subject with a polynucleotide encoding a Gₛα protein. Alternatively, a cell can be transfected in vitro with a vector designed to insert, by homologous recombination, a new, active promoter upstream of the transcription start site of the naturally occurring endogenous Gₛα gene in the cell's genome. Such methods, which “switch on” an otherwise largely silent gene, are well known in the art. After selection and expansion of a cell that expresses Gₛα at a desired level, the transfected or transduced cells are then returned to the subject. The cells can be any of a wide range of types including, without limitation, neral cells, hemopojetic cells (e.g., bone marrow cells, macrophages, monocytes, dendritic cells, T cells, or B cells), fibroblasts, epithelial cells, endothelial cells, keratinocytes, or muscle cells. Such cells act as a source of the Gₛα protein for as long as they survive in the subject.

The ex vivo methods include the steps of harvesting cells from a subject, culturing the cells, transducing them with an expression vector, and maintaining the cells under conditions suitable for expression of the Gₛα gene. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the Gₛα gene. The cells may then be injected or implanted into the subject.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Materials and Methods

1. cDNA Subtraction Analysis

Total mRNA was isolated from the WAT of C/EBPαβ and control mice and converted to cDNA. cDNA subtraction analysis was performed using a commercial kit based on PCR amplification (PCR-Select cDNA Subtraction Kit, Clontech, USA). The subtracted cDNA was then divided into two pools, one containing cDNA species corresponding to mRNA molecules that are present or enhanced in the WAT of C/EBPαβ mice, and the other containing cDNA species corresponding to mRNA molecules that are absent or reduced in the WAT of C/EBPαβ mice. The subtracted cDNA species were then subjected to automated sequencing.

2. Northern Blotting Confirmation

The selected cDNA species were used to probe total RNA isolated from the WAT of C/EBPαβ mice. Total RNA was resolved in 1% formaldehyde agarose gel by electrophoresis, blotted onto a nylon membrane, and hybridized to ³²P-labelled cDNA probes.

3. Adenoviral Expression System

Once the expression pattern of a selected mRNA species was confirmed, corresponding full-length cDNA was cloned. For human Gₛα sequence, see GenBank accession No. X56009; for mouse Gₛα sequence, see GenBank accession No. M13964. The full-length cDNA was then subcloned into an adenoviral transfer vector that contains a jellyfish green fluorescence protein (GFP) for visualizing and monitoring the infection efficiency. The subcloned cDNA was expressed under the control of the CMV promoter for ubiquitous expression in mammalian cells. The adenoviral transfer vector containing the interested cDNA species was then used to co-transform E. coli with a modified adenoviral genome (both EI and EIII genes deleted to abolish the viral replication ability). The transformed E. coli was selected for harboring the correctly recombined adenoviral genome where the desired cDNA fragment has integrated. The recombinant adenoviral genome was then extracted from the E. coli clones and transfected into 293 HEK cells to produce adenoviral particles according to the manufacturer's protocol (AdEasy kit, QBI, Germany). The culture media containing adenovirus particles released from the infected cells were used directly to infect cultured fat cells.

4. Mouse Fat Cell Culture

A mouse pre-adipocyte cell line, 3T3-L1, was cultured and induced into fat-laden adipocyte by allowing cells to grow into confluency. Insulin, Dex and IBMX were then added to the culture media for 4 days to induce adipogenesis (Yeh, et al. (1995) Genes & Dev. 9, 168-181).

5. Human Fat Cell Culture

Two grams of abdominal fat biopsy from a human patient who underwent surgical treatment for other illness was treated with 0.1% collagenase and gently shacked at 37° C. for 1 hour to dissociate the cells. Collagenase activity was then neutralized with DMEM containing 10% FBS, and the cells were centrifuged at 1,200 g for 10 min to obtain a cell pellet. The cell pellet was resuspended in 160 mM NH₄Cl and incubated at room temperature for 10 min to lyze red blood cells. The pre-fat cells were then incubated overnight at 37° C., 5% CO₂ in DMEM/F12 (1:1) containing 10% FBS and antibiotics. After overnight culture, unattached cells were removed, and the culture medium was changed every 2 days during the culture period (Zuk, et al. (2001) Tissue Engineering 7, 211-228). The method used for differentiating mouse 3T3-L1 into adipocyte was used with slight modification to induce human fat cell conversion.

6. Adenovirus Infection

Once the fat-laden fat cells were produced (2 days after adipogenesis started), the old medium was removed, and 100 μl (for a well of 24-well culture plate) of medium containing adenovirus particles was added to the fat cells and incubated at 37° C. for 1 hour. 0.5 ml of fresh fat cell medium was then added to each well, and the fat cells were continually cultured. The culture medium was changed every 2 days during the culture period.

7. GFP Monitoring

The adenoviral vectors, each carrying one interested cDNA species, also contain a GPF protein marker that would emit green fluorescence under fluorescence microscope. Any fat cells that have been infected with an adenovirus would emit green fluorescence, and those that have not been infected would emit no fluorescence. Accordingly, the fat cells infected with and without an adenovirus can be distinguished and compared for their status on lipid accumulation.

Results

1. Screening for MRNA Species Differentially Expressed in WAT of C/EBPαβ Mouse

In the WAT of C/EBPαβ mice, mitochondrial content was significantly increased both in size and number when compared to that in the WAT of control mice. In addition, the metabolic activity in the WAT of C/EBPαβ mice was significantly higher as measured by the cytochrome c oxidase activity and by the expression levels of several factors crucial in energy oxidation. To find out whether this increase of mitochondrial content in the WAT of C/EBPαβ mice is due to increased expression of gene(s) related to mitochondrial biogenesis and are regulated by the C/EBP proteins, mRNA species whose expression levels are enhanced or induced specifically in the WAT of C/EBPαβ mice and whose expression levels may be important in mitochondrial biogenesis in fat cells were identified.

A pool of subtracted cDNAs containing MRNA species expressed only or more in the WAT of C/EBPαβ mice were cloned and then sequenced. Furthermore, for some mRNA species including the G protein stimulatory α subunit (Gₛα) mRNA, Northern blot analysis was performed to confirm their expression pattern in the WAT of C/EBPαβ mice.

2. Construction of Adenoviral Expression Vectors Containing Human Full-length Gₛα, Mouse Full-length Gₛα, and truncated Gₛα

To study the role of Gₛα in energy oxidation in WAT, an expression vector carrying the Gₛα coding region was constructed to introduce and express Gₛα in fat cells. Mature fat cells are highly differentiated and quiescent. Thus, they cannot be transfected efficiently by commonly used methods, such as calcium phosphate and lipofectin method. Therefore, adenoviral delivery system was employed to express Gₛα in fat cells. Adenovirus with EI and EIII genes deleted, although incapable of replicating, can infect mammalian cells with high efficiency, and is therefore widely used as a vehicle to deliver genes into mammalian cells.

Full-length cDNAs for both human and mouse Gₛα were cloned into pAd.track.CMV (He, et al. (1998) Proc. Natl. Acad. Sci. USA 95, 2509-2514), a transfer vector plasmid containing a GFP gene driven by a CMV promoter that is active in most mammalian cells. Recombinant adenovirus carrying a Gₛα gene was then generated according to the manufacturer's protocol (AdEasy, QBI; and He, et al. (1998) Proc. Natl. Acad. Sci. USA 95, 2509-2514). Since the expression of the Gₛα gene is also under the control of the CMV promoter, fat cells infected with the recombinant adenovirus and emitting green fluorescence should also express Gₛα. The expression of the Gₛα protein in the infected 293 HEK cells was confirmed by Western blot analysis using an antibody specific against Gₛα.

In addition to the adenovirus that carries the full-length Gₛα, a recombinant adenovirus that carries a truncated mouse Gₛα lacking 59 amino acids at the N-terminus was also generated. This truncated Gₛα is inactive, due to the lack of the N-terminal domain required for interacting with dimeric Gαγ for membrane targeting (Evanko, et al. (2000) J. Biol. Chem. 275, 1327-1336). This truncated Gₛα was used as a control for the experiment described below.

3. Prevention of Lipid Accumulation in Fat Cells Infected with Human or Mouse Full-Length Gₛα Gene

Expression of Gₛα was specifically increased in the fat cells of WAT in C/EBPαβ mice. To find out the effects of Gₛα on fat accumulation in fat cells, human and mouse fat cells cultured from human pre-adipocytes isolated from the abdominal fat tissue of a patient and mouse pre-adipocytes, 3T3-L1were infected with recombinant adenoviruses carrying human and mouse Gₛα, respectively. Green fluorescence was emitted from cells that were infected with the recombinant adenoviruses when observed under a fluorescence microscope.

Lipid droplets inside a fat cell are visible under a reverse light-microscope. Thus the degree of lipid accumulation (i.e., the number and size of droplets) in fat cells can be readily determined under a microscope. Under the culture condition, lipid accumulation in normal fat cells (both human and mouse) steadily increased as shown by the change of lipid droplets in number and size once every 24 hours. By contrast, fat accumulation appeared to be regressive in cells infected with the recombinant adenovirus carrying either the human or mouse full-length Gₛα. On the other hand, fat cells infected with the recombinant adenovirus carrying the truncated, inactive form of Gₛα, continued to accumulate lipids as non-infected fat cells. These results suggest that the effect of Gₛα on fat accumulation are conserved from mouse to human, because both the human and mouse Gₛα are effective in preventing fat accumulation in fat cells.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1-6. (canceled)

7. A method of identifying a compound for treating a disorder in fat metabolism, the method comprising:
contacting a compound with a cell, and
determining a Gsa expression level in the cell,
wherein the Gsa expression level in the presence of the compound, if different from that in the absence of the compound, indicates that the compound is a candidate for treating a disorder in fat metabolism.

8. The method of claim 7, wherein the cell is a fat cell.

9. The method of claim 8, wherein the cell is a white adipose cell.

10. A method of identifying a compound for treating a disorder in fat metabolism, the method comprising:
contacting a compound with a cell, and
determining a Gsa activity level in the cell,
wherein the Gsa activity level in the presence of the compound, if different from that in the absence of the compound, indicates that the compound is a candidate for treating a disorder in fat metabolism.

11. The method of claim 10, wherein the cell is a fat cell.

12. The method of claim 11, wherein the cell is a white adipose cell.

13-23. (canceled)
